SEQUENCE LISTING (5 sequences)

SEQ ID NO: 1 - DNA, 2456 bp, Homo sapiens
gcaagcacggaacaagctgagacggatgataatatggatacaaaatctattctagaagaacttcttctcaaaagatcacagcaaaagaagaaaatgtcaccaaataattacaaagaacggctttttgttttgaccaaaacaaacctttcctactatgaatatgacaaaatgaaaaggggcagcagaaaaggatccattgaaattaagaaaatcagatgtgtggagaaagtaaatctcgaggagcagacgcctgtagagagacagtacccatttcagattgtctataaagatgggcttctctatgtctatgcatcaaatgaagagagccgaagtcagtggttgaaagcattacaaaaagagataaggggtaacccccacctgctggtcaagtaccatagtgggttcttcgtggacgggaagttcctgtgttgccagcagagctgtaaagcagccccaggatgtaccctctgggaagcatatgctaatctgcatactgcagtcaatgaagagaaacacagagttcccaccttcccagacagagtgctgaagatacctcgggcagttcctgttctcaaaatggatgcaccatcttcaagtaccactctagcccaatatgacaacgaatcaaagaaaaactatggctcccagccaccatcttcaagtaccagtctagcgcaatatgacagcaactcaaagaaaatctatggctcccagccaaacttcaacatgcagtatattccaagggaagacttccctgactggtggcaagtaagaaaactgaaaagtagcagcagcagtgaagatgttgcaagcagtaaccaaaaagaaagaaatgtgaatcacaccacctcaaagatttcatgggaattccctgagtcaagttcatctgaagaagaggaaaacctggatgattatgactggtttgctggtaacatctccagatcacaatctgaacagttactcagacaaaagggaaaagaaggagcatttatggttagaaattcgagccaagtgggaatgtacacagtgtccttatttagtaaggctgtgaatgataaaaaaggaactgtcaaacattaccacgtgcatacaaatgctgagaacaaattatacctggcagaaaactactgttttgattccattccaaagcttattcattatcatcaacacaattcagcaggcatgatcacacggctccgccaccctgtgtcaacaaaggccaacaaggtccccgactctgtgtccctgggaaatggaatctgggaactgaaaagagaagagattaccttgttgaaggagctgggaagtggccagtttggagtggtccagctgggcaagtggaaggggcagtatgatgttgctgttaagatgatcaaggagggctccatgtcagaagatgaattctttcaggaggcccagactatgatgaaactcagccatcccaagctggttaaattctatggagtgtgttcaaaggaataccccatatacatagtgactgaatatataagcaatggctgcttgctgaattacctgaggagtcacggaaaaggacttgaaccttcccagctcttagaaatgtgctacgatgtctgtgaaggcatggccttcttggagagtcaccaattcatacaccgggacttggctgctcgtaactgcttggtggacagagatctctgtgtgaaagtatctgactttggaatgacaaggtatgttcttgatgaccagtatgtcagttcagtcggaacaaagtttccagtcaagtggtcagctccagaggtgtttcattacttcaaatacagcagcaagtcagacgtatgggcatttgggatcctgatgtgggaggtgttcagcctggggaagcagccctatgacttgtatgacaactcccaggtggttctgaaggtctcccagggccacaggctttaccggccccacctggcatcggacaccatctaccagatcatgtacagctgctggcacgagcttccagaaaagcgtcccacatttcagcaactcctgtcttccattgaaccacttcgggaaaaagacaagcattgaagaagaaattaggagtgctgataagaatgaatatagatgctggccagcattttcattcattttaaggaaagtaggaaggcataagtaattttagctagtttttaatagtgttctctgtattgtctattatttagaaatgaacaaggcaggaaacaaaagattcccttgaaatttagatcaaattagtaattttgttttatgctgctcctgatataacactttccagcctatagcagaagcacattttcagactgcaatatagagactgtgttcatgtgtaaagactgagcagaactgaaaaattacttattggatattcattcttttctttatattgtcattgtcacaacaattaaatatactaccaagtacagaaatgtggaaaaaaaaaaccg

SEQ ID NO: 2 - protein, 651 aa, Mus musculus
MESKSILEELLLKKSQQKKKMSPNNYKERLFVLTKTSLSYYEYDKMKRGSRKGSIEIKKIRCVEKVNLEEQTPVERQYPFQIVYKDGLLYVYASNEESRCQWLKALQKEIRGNPHLLIKYHSGFFVDGKFLCCQQSCKAAPGCTLWEAYADLHIAISDEKHRAPTFPERLLKIPRAVPVLKMDASSSGAILPQYDSYSKKSCGSQPTSNIRYIPREDCPDWWQVRKLKSEEDIACSNQLERNIASHSTSKMSWGFPESSSSEEEENLHAYDWFAGNISRSQSEQLLRQKGKEGAFMVRNSSQMGMYTVSLFSKAVNDKKGTVKHYHVHTNAENKLYLAENYCFDSIPKLIHYHQHNSAGMITRLRHPVSTKANKVPVSVALGSGIWELKREEITLLKELGNGQFGVVQLGQWKGQYDVAVKMIKEGAMSEDEFFQEAQTMMKLSHPKLVKFYGVCSKKYPIYIVTEYITNGCLLNYLKSHGKGLESCQLLEMCYDVCKGMAFLESHQFIHRDLAARNCLVDRDLSVKVSDFGMTRYVLDDQYVSSVGTKFPVKWSAPEVFHYFKYSSKSDVWAFGILMWEVFSLGKQPYDLYDNSEVVVKVSQGHRLYRPQLASDTIYQIMYSCWHELPEKRPTFQQLLSAIEPLREQDKP

SEQ ID NO: 3 - protein, 675 aa, Homo sapiens
MDTKSILEELLLKRSQQKKKMSPNNYKERLFVLTKTNLSYYEYDKMKRGSRKGSIEIKKIRCVEKVNLEEQTPVERQYPFQIVYKDGLLYVYASNEESRSQWLKALQKEIRGNPHLLVKYHSGFFVDGKFLCCQQSCKAAPGCTLWEAYANLHTAVNEEKHRVPTFPDRVLKIPRAVPVLKMDAPSSSTTLAQYDNESKKNYGSQPPSSSTSLAQYDSNSKKIYGSQPNFNMQYIPREDFPDWWQVRKLKSSSSSEDVASSNQKERNVNHTTSKISWEFPESSSSEEEENLDDYDWFAGNISRSQSEQLLRQKGKEGAFMVRNSSQVGMYTVSLFSKAVNDKKGTVKHYHVHTNAENKLYLAENYCFDSIPKLIHYHQHNSAGMITRLRHPVSTKANKVPDSVSLGNGIWELKREEITLLKELGSGQFGVVQLGKWKGQYDVAVKMIKEGSMSEDEFFQEAQTMMKLSHPKLVKFYGVCSKEYPIYIVTEYISNGCLLNYLRSHGKGLEPSQLLEMCYDVCEGMAFLESHQFIHRDLAARNCLVDRDLCVKVSDFGMTRYVLDDQYVSSVGTKFPVKWSAPEVFHYFKYSSKSDVWAFGILMWEVFSLGKQPYDLYDNSQVVLKVSQGHRLYRPHLASDTIYQIMYSCWHELPEKRPTFQQLLSSIEPLREKDKH

SEQ ID NO: 4 - protein, 20 aa, Homo sapiens
CNLHTAVNEEKHRVPTFPDR

SEQ ID NO: 5 - DNA, 23 bp, Homo sapiens
tgttgctgttaggatgatcaagg

(19) United States
(12) Patent Application Publication
(10) Pub. No.: US 2002/0173481 A1
(43) Pub. Date: Nov. 21, 2002

(54) Regulation of vascular endothelium using BMX tyrosine kinase

(76) Inventors: Niklas Ekman, Helsinki (FI); Elena Arighi, Helsinki (FI); Imre Vastrik, London (GB); Luca Tamagnone, Torino (IT); Kari Alitalo, Espoo (FI)

Correspondence Address: David A. Gass, MARSHALL, GERSTEIN & BORUN, Sears Tower, 233 S. Wacker Drive, Suite 6300, Chicago, IL, 60606-6357, US

(21) Appl. No.: 10/186,399

(22) Filed: Jul. 1, 2002

Related U.S. Application Data
(63) Continuation of application No. 09/538,445, filed on Mar. 29, 2000.
Application No. 09/538,445 is a continuation of application No. 09/104,863, filed on Jun. 25, 1998, now abandoned.
Application No. 09/104,863 is a continuation-in-part of application No. 08/320,432, filed on Oct. 7, 1994, now abandoned.

(51) Int. Cl. A61K 48/00; A61K 38/20; A61K 38/18

(52) U.S. Cl. 514/44; 424/85.2; 514/12

(57) ABSTRACT

Vascular endothelia are subject to atherosclerotic and arteriostenotic effects transduced by molecules, such as thrombin, IL-3 and VEGF which can lead to vessel occlusion or stenosis. An endothelial signaling pathway involving the Bmx tyrosine kinase contributes to normal endothelial nonthrombogenic, inflammatory and growth conditions of arterial vessels, and regulation of the pathway can treat or prevent pathological conditions in the vessel walls.

### CROSS-REFERENCE

This application is a continuation in part of U.S. patent application Ser. No. 08/320,432, filed Oct. 7, 1994 entitled “Cytoplasmic Tyrosine Kinase,” which is hereby incorporated by reference in its entirety.

### BACKGROUND OF THE INVENTION

The field of the invention is control of vascular endothelium. The vascular endothelium controls important properties, such as inflammatory responses, the regulation of a nonthrombogenic surface and responses of the vessel wall to products of platelet activation on the vessel wall, and recanalization of vascular occlusions by thrombi. Arteriosclerosis is considered to arise in part because of failure of the nonthrombogenic vessel surface, and preferred sites for arteriosclerosis are arterial bifurcations and sites of high shear pressure and turbulent blood flow. At such sites, microaggregates of platelets are thought to form and be activated, resulting in the release of substances that promote arteriosclerosis. Clotted blood or fibrin deposits often form in damaged vessel walls at such sites. Thrombin is a proteolytic enzyme of the blood clotting cascade, which has unique effects on the formation and deposition of fibrin clots and on the properties of the vascular endothelium of the vessel wall. Thrombin is highly stimulatory for endothelial cell growth (mitosis) and migration in culture and it causes cytoskeletal changes in the stimulated cells (Van Obberghen-Schilling et al. (1995) Annals of the New York Academy of Sciences 766:431-41).

Platelets play an essential role in acute coronary syndromes such as unstable angina and myocardial infarction (Handin (1996) New England Journal of Medicine 334(17):1126-7). In addition, platelet inhibitory trials suggest that activation of platelets plays an important role in the natural history of coronary artery disease in general as well as in coronary bypass graft disease (Le Breton et al. (1996) Journal of the American College of Cardiology 28(7):1643-51). Although under normal conditions platelets circulate in the blood an inactivated state, under pathological conditions, the cells can be stimulated to release a variety of substances such as serotonin and thromboxane A2, which evoke potent vasoconstriction and further activation of platelets. In patients with coronary artery disease, local platelet activation commonly occurs and contributes to local vasospasm, thrombus formation, and eventually vascular occlusion. Thrombin is a potent platelet activator and has been implicated in thrombotic coronary artery occlusion and in particular in vascular reocclusion after coronary thrombolysis and angioplasty. Antithrombin therapy appears to prevent vascular reocclusion after thrombolysis in an animal model (Gallo et al. (1998) Circulation 97(6):581-8).

In addition to platelets, thrombin activates endothelial cells of human arteries (Garcia et al. (1995) Blood Coagulation & Fibrinolysis 6(7):609-26). Endothelial cells manifest antithrombotic activity by releasing vasoactive substances with antiplatelet activities such as the endothelium-derived nitric oxide and prostacyclin. Platelet-derived substances (i.e., adenine nucleotides, serotonin) and thrombin, on the other hand, cause endothelium-dependent relaxation, at least in certain blood vessels. Hence, platelet-derived substances such as serotonin, ADP, and ATP as well as thrombin can activate platelets and stimulate the release of nitric oxide and prostacyclin from the endothelium. Thus, depending on the functional status of the endothelium, the thrombin-regulated substances should differentially affect the vessel wall. Since endothelial dysfunction, and in particular a decreased release of nitric oxide and prostacyclin, occurs in coronary artery disease, these mechanisms may have important implications in unstable angina and myocardial infarction (Yang et al. (1994) Circulation 89(5):2266-2272; herein incorporated by reference).

The vascular endothelium of arteries is subject to physiological stresses, such as high intravascular pulsatile pressure and shear stress, which have been shown to control gene expression in endothelial cells (Gimbrone et al. (1997) Journal of Clinical Investigation 100(11 Suppl):S61-5; herein incorporated by reference). Such influences on the endothelial cells, including readjustment of gene expression to stressful conditions, are controlled by signals from the extracellular fluid and pericellular matrix via specific cell surface receptors, such as hemopoietin-cytokine receptors, receptor tyrosine kinases and G-protein coupled receptors (Guidebook to Cytokines and Their Receptors, Nicos A. Nicola (Ed.), Oxford University Press, 1994; herein incorporated by reference). For example, shear stress has been shown to lead to changes in the TGFβ-SMAD signal transduction in endothelial cells, which is considered, among other things, to lead to changes in the biosynthesis of extracellular matrix components by the cells (Topper et al. (1997) Proceedings of the National Academy of Sciences of the United States of America 94(17):9314-9).

Recent evidence shows that there are endothelial cell specific growth factors and receptors that may be primarily responsible for the stimulation of endothelial cell growth, differentiation and certain differentiated functions. The best studied of these is vascular endothelial growth factor (VEGF). VEGF is a dimeric glycoprotein of disulfide-linked 23 kD subunits. Other reported effects of VEGF include the mobilization of intracellular calcium, the induction of plasminogen activator and plasminogen activator inhibitor-1 synthesis, stimulation of nitric oxide release and hexose transport in endothelial cells, and promotion of monocyte migration in vitro. VEGF was originally purified from several sources on the basis of its mitogenic activity toward endothelial cells, and also by its ability to induce microvascular permeability, hence it is also called vascular permeability factor (VPF).

Two high affinity receptors for VEGF have been characterized: VEGFR-1/Flt-1 (fms-like tyrosine kinase-1) and VEGFR-2/KDR/FIk-1 (kinase insert domain containing receptor/fetal liver kinase-1). Those receptors have seven immunoglobulin-like loops in their extracellular domain, and they possess a longer kinase insert than normally observed in other receptors of this family. The expression of VEGF receptors occurs mainly in vascular endothelial cells, although some may be present on hematopoietic progenitor cells, monocytes, osteoblasts, corneal endothelial cells, pericytes, leydig and sertoli cells and melanoma cells. Only endothelial cells have been reported to proliferate in response to VEGF, and endothelial cells from different vessels show different responses (see e.g., review by Korpelainen and Alitalo (1998) Current Opinion in Cell Biology 10:159-164; Jeltsch et al. (1997) Science 276:1423-1425; herein incorporated by reference). Thus, the signals mediated through VEGF receptors appear to be cell type specific. The VEGF-related placenta growth factor (PlGF) was recently shown to bind to VEGFR-1 with high affinity. PlGF was able to enhance the mitogenic or growth factor activity of VEGF, and stimulated DNA synthesis in capillary endothelial cells (Ziche et al. (1997) Laboratory Investigation 76(4):517-31; herein incorporated by reference). Naturally occurring VEGF/PlGF heterodimers were nearly as potent mitogens as VEGF homodimers for endothelial cells (Cao et al., (1996) J. Biol. Chem., 271:3154-62; herein incorporated by reference).

Cytokines may also activate important signals for the reprogrammming of vascular endothelial cells. For example, IL-3 is a haemopoietic growth factor which stimulates the production and functional activity of various blood cell types. Recent evidence suggests that the target cell population of IL-3 is not restricted to haemopoietic cells as previously thought, but vascular cells such as endothelial cells also express receptors for and respond to this cytokine. IL-3 was found to regulate endothelial responses related to inflammation, immunity and haemopoiesis (Korpelainen et al. (1996) Immunology & Cell Biology 74(1):1-7; herein incorporated by reference). Immune mechanisms also play a major role in the development of arteriosclerosis (Yokota and Hansson (1995) J. Internal Medicine 238(6):479-89). Thus the function of IL-3 may be of clinical importance, as IL-3 can be used in bone marrow reconstitution following cancer therapy.

Another example is thrombin, which is activated by blood clotting, has strong effects on endothelial cells directly, via an endothelial G-protein coupled thrombin receptor, and via platelet activation with the resulting release of effectors from platelet alpha granules.

Certain of such signals, such as those via the cytokine, receptor tyrosine kinase and G-protein coupled receptors, may be transmitted via cytoplasmic tyrosine kinases.

Tyrosine protein kinases (TKs) are essential components of signal transduction in endothelial cells. Several of the TKs function as transmembrane receptors, transducing signals from growth factors to the cytoplasm (Mustonen and Alitalo (1995) J. Cell Biol. 129: 895-898; herein incorporated by reference). The extracellular domains of the receptor TKs are responsible for ligand binding, while the intracellular TK domains transmit the activation signals through phosphorylation of cellular polypeptides. Five different endothelial cell receptor TKs are known, encoded by two different gene families (Mustonen and Alitalo, supra). Before our studies on Bmx, non-receptor tyrosine kinases relatively specific for endothelial cells had not been reported.

The Bmx TK belongs to the so-called Btk subfamily (Vihinen and Smith (1996) Crit. Rev. Immunol. 16(3):251-275). The four proteins encoded by members of this gene family share substantial homology, including typical SH2 and SH3 domains upstream of the TK domain. A special feature of these TKs is a so-called pleckstrin homology (PH) domain in the N-terminal region (Musacchio et al. (1993) TIBS 18:343-348). Several of these non receptor TKs have been shown to be expressed in various cultured hematopoietic cell lines. The Tec TK is expressed in all murine hematopoietic cell lines examined (Mano et al. (1990) Oncogene 5:1781-1786). The Tec kinase is activated by multiple cytokine receptors in the hematopoietic cells and by thrombin and integrin signals in blood platelets (Hamazaki et al. (1998) Oncogene 16:2773-2779). Itk (Gibson et al. (1993) Blood 82:1561-1572) and Btk (de Weers et al. (1993) Eur J immunol. 23:3109-3114) are selectively expressed at certain stages of lymphocyte development and the expression of the Txk TK has been assigned to T-cells (Sommers et al. (1995) Oncogene 11:245-251).

The Bmx TK gene was isolated while screening for novel TK genes expressed in human bone marrow (Tamagnone et al. (1994) Oncogene 9:3683-3688; herein incorporated by reference). Because the gene was mapped to chromosome X at Xp22.2, it was called Bmx, Bone Marrow tyrosine kinase gene in chromosome X. The X chromosome is the location of at least one known tyrosine kinase gene-linked disease in humans, the X-linked agammaglobulinemia. Several other human mutations are known in genes located in the X-chromosome, which lead to disease in a hemizygous position in males, because of the lack of another X-chromosome and thus in many cases the lack of a healthy allele in male cells. In comparison with other Btk family members, Bmx lacks the so-called P-X-P motifs but has extra peptides in between PH and SH3 and SH2 domains. The SH3 sequence does not conform precisely to the described consensus.

### SUMMARY OF THE INVENTION

The invention can be used to treat or prevent arteriostenosis, or narrowing of the lumen of an artery or the heart, e.g., by arteriosclerosis (hardening of the wall of an artery), in a patient by regulating the activity of Bmx tyrosine kinase in arterial endothelial cells and/or endocardial endothelial cells in a manner sufficient to inhibit inflammatory responses, growth signals, or to reduce thrombogenic tendencies or properties of such endothelia. The inhibition of Bmx in the endothelium will inhibit the intimal migration and growth of associated smooth muscle cells from the tunica media of arteries.

In preferred embodiments, the arteriostenosis to be treated arises from arteriosclerosis or vasospasm; and preferably the Bmx tyrosine kinase regulation occurs by administering to the patient to be treated an agent capable of inhibiting Bmx tyrosine kinase activity. By “patient” is meant an animal, preferably a mammal, more preferably a human. By “agent” is meant a ligand, drug, chemical, compound, nucleic acid, or any other substance which, directly or indirectly, is capable of acting on the desired target. For example, if Bmx tyrosine kinase activity involves the tyrosine phosphorylation of another protein, an effective agent would be one administered to the patient which prevents Bmx from phosphorylating another protein due to interference with any link in the upstream cascade of molecular events within a cell which leads to such phosphorylation, or impedes the phosphorylation directly.

In preferred embodiments, a tyrosine kinase inhibitor or BMX antisense cDNA is the agent used to inhibit the Bmx tyrosine kinase activity. Alternatively, genetically modified BMX cDNA is used to express altered Bmx protein; this Bmx or the BMX cDNA is molecularly or functionally distinguishable from naturally occurring BMX DNA or Bmx protein. By “molecularly or functionally distinguishable” is meant that one of skill in the art using art-known techniques (e.g., nucleic acid or amino acid sequencing, or in vitro phosphorylation assays) could determine that the Bmx is not of natural origin or is not the exact molecule normally found in nature in the animal in question.

Preferably, the region of endocardial or arterial endothelium or smooth muscle targeted is an area of turbulent vascular flow, such as, e.g., the chambers of the heart, areas of vessel bifurcation, or diseased regions (e.g., those with developing atherosclerotic plaques). The agent can be directly (e.g., via a catheter) or indirectly (e.g., via agents introduced into the body or circulation alone, in liposomes, vectors, etc.) administered to the vascular endothelium or endocardium of the patient.

In other preferred embodiments, the regulation of Bmx tyrosine kinase activity is via a ligand which is able to bind to a cell surface receptor (e.g., on an endothelial cell) which, when bound, elicits a molecular (e.g., phosphorylation) cascade linked to Bmx tyrosine kinase. By “ligand” is meant an extracellular molecule which specifically binds to a receptor (e.g., a vascular endothelial growth factor receptor such as VEGFR-1/Flt-1 or VEGFR-2/KDR/Flk-1) and either blocks or elicits an intracellular cascade, leading to increased or decreased activity of the target molecule. Particularly preferred ligands of the invention are interleukin-3 (IL-3) and vascular endothelial growth factor (VEGF).

Particularly preferred embodiments include regulating Bmx tyrosine kinase activity in endocardial and/or arterial endothelial cells and/or arterial smooth muscle cells in order to inhibit, reduce, or prevent a thrombotic (clotting), mitotic (cell division) or inflammatory (e.g., cytokine mediated) effects in such cells or surrounding smooth muscle cells. Inhibiting the effects of the compounds thrombin, IL-3, and VEGF is particularly preferred.

In another embodiment, regulation of Bmx is in order to accelerate the re-endothelialization of damage endothelium after surgery in the vessel wall, such as balloon angioplasty or the implantation of a vascular prosthesis. By “re-endothelialization is meant the regrowth of healthy endothelial lining of a vessel damaged by disease or trauma (e.g., surgical procedure.) Yet another application is for the relaxation of arterial smooth muscle cells adjacent to the endothelial cells, via regulation of endothelial cell Bmx kinase activity.

A preferred embodiment of the invention is a method of identifying agents which affect a Bmx tyrosine kinase signaling pathway, the method comprising applying an agent or agents to be tested to the tissue of a transgenic animal or to such an animal itself, the cells of the animal having a genetic defect in the Bmx encoding region of the genome, the defect causing an abnormal Bmx signaling pathway (e.g., because of a defective protein or a defect in expression of the protein), and a suitable agent being one which functionally restores at least one step in the abnormal pathway. By “step in the pathway” is meant DNA transcription, RNA translation, or protein function, e.g., proper cleavage or conformation/folding, enzymatic properties (phosphorylation), or ability to participate in a molecular cascade (e.g., to be phosphorylated).

Another preferred embodiment is a method for diagnosing a human defect or disease associated with Bmx dysfunction (e.g., protein dysfunction or transcriptional/translational dysfunction of the nucleic acids) caused by a mutation in the BMX gene on chromosome X of a patient, the method comprising an assay of the BAM gene and an analysis of the assay results sufficient to detect the mutation. Any assays known in the art for detecting mutations or gene defects or abnormalities may be used, such as restriction digests, PCR assays, nucleic acid sequencing, Southern or Northern blotting, hybridization of labeled oligonucleotides to the gene, or any suitable commercial kits (e.g., technology such as Incstar's Gen-eti-k DEIA kit for enzyme immunoassay detection of double stranded DNA (detecting hybridized probe and template DNA)).

Terms used herein are to be given their art-known meaning. For example, the term “antisense” means an RNA or DNA sequence which is sufficiently complementary to a particular target RNA or DNA molecule for which the antisense RNA or DNA is directed to cause molecular hybridization between the antisense RNA or DNA and the target RNA or DNA such that transcription or translation of RNA or protein is inhibited. Such hybridization occurs in vivo, that is, inside the cell. The action of the antisense molecule results in specific inhibition of gene expression in the cell. (See: Alberts, B. et al., Molecular Biology of the Cell, 2nd Ed., Garland Publishing, Inc., New York. N.Y. (1989), in particular, pages 195-196; herein incorporated by reference.) The antisense molecule may be comprised of 10 or more naturally or non-naturally occurring nucleotides (e.g., an example of a non-naturally occurring nucleotide could include molecules with enhanced hybridization affinity such as described in U.S. Pat. No. 5,432,272, herein incorporated by reference). “Arteriostenosis” means a narrowing, hardening, or occlusion of the caliber of an artery, either temporarily, through e.g., vasoconstriction, or permanently, through e.g., arteriosclerosis. (See, e.g., Stedmans Medical Dictionary, 25th Ed., Williams & Wilkins, Baltimore, Md., (1990)).

### DESCRIPTION OF THE DRAWINGS

FIG. 1A: The mouse Bmx cDNA structure and the isolated clones are shown schematically;

FIG. 1B: A comparison of the deduced amino acid sequences of mouse and human Bmx genes is shown;

FIG. 1C: mRNA signals obtained from the heart and lung;

FIG. 2: In 10.5 and 12.5 day p.c. mouse embryos the Bmx autoradiographic signals decorated the heart endocardium;

FIG. 3: Transverse sections of the thoracic cavity show strong Bmx mRNA signals present in the aortal endothelial cells and subclavian arteries as well as in the intervertebral arteries (FIGS. 3A and B). Signal was seen also in the umbilical arteries (FIGS. 3C and 3D). Such signals were considerably weaker in the aortic endothelium of 16.5 day mouse embryos (FIGS. 3E and 3F). The Bmx sense probe did not give a signal in any of these sections (FIGS. 3G and 3H). Autoradiographic signals were obtained from the heart endocardium of the left ventricle and from aorta (FIGS. 3J and 3K). The coronary arteries showed a weak but definitive hybridization signal (FIGS. 3L and 3M);

FIG. 4: Co-expression of Jak1 and Jak2 induces phosphorylation of kinase dead Bmx;

FIG. 5: Comparison of Bmx phosphorylation level after cotransfection with Epo, GCSF, Flt1, KDR or Flt4 receptors;

FIG. 6: Flt1, GCSF and Epo receptors increase Bmx in vitro kinase activity;

FIG. 6A: 293T cells were transfected with Bmx together with indicated receptors. Bmx was immunoprecipitated from cell lysates using anti-HA-Ab and kinase activity was mesured as described in materials and methods;

FIG. 6B: Kinase reactants were electrophoresed in 7.5% SDS-PAGE. The gel was dried and Bmx autokinase activity was visualised by exposure on film;

FIG. 6C: A part of the same immunoprecipitants were electrophoresed in 7.5% SDS-PAGE, transferred to nitrocellulose membrane and immunoblotted with anti-Bmx-Ab;

FIG. 7: Flt1 stimulates Bmx kinase activity, but other VEGF receptors do not;

FIG. 7A: 293T cells were transfected with Bmx together with indicated receptors; Bmx was immunoprecipitated from cell lysates using anti-HA-Ab and kinase activity was measured as described in materials and methods;

FIG. 7B: Kinase reactants were electrophoresed in 7.5% SDS-PAGE. The gel was dried and Bmx autokinase activity was visualised by exposure on film;

FIG. 7C: A part of the same immunoprecipitants were electrophoresed in 7.5% SDS-PAGE, transferred to nitrocellulose membrane and immunoblotted with anti-Bmx-Ab;

FIG. 8: Endogenous receptors for interleukin 3 and GCSF increase Bmx kinase activity. 32Dcl3 cells expressing Bmx or kinase dead Bmx were starved overnight in IL-3 free media and then stimulated with IL-3 or GCSF. Bmx was immunoprecipitated from cell lysates using anti-HA-Ab and kinase activity was measured;

FIG. 9: Activation of endogenous Bmx of endothelial cells;

FIG. 9A: Human Umbilical Vein Endothelial Cells (HUVECs) were starved overnight and then stimulated with indicated factors with or without inhibitors of PI-3 kinase. Bmx was immunoprecipitated from cell lysates using anti-HA-Ab and kinase activity was measured;

FIG. 9B: Kinase reactants were electrophoresed in 7.5% SDS-PAGE. The gel was dried and Bmx autokinase activity was visualised by exposure on film;

FIG. 9C: Shown in panel C is an immunoblot from total cell lysates electrophoresed in 6% SDS-PAGE blotted with endothelial specific anti-CD31 Ab;

FIG. 10: Knock-in type gene targeting of mouse Bmx in embryonic stem cells.

FIG. 10A: Schematic representation of the targeting construct and the wild-type and targeted mouse Bmx locus. Using this strategy, the first coding exon has been replaced by the LacZ and neomycin casettes by homolougus recombination. E=Eco RI, B=Bam HI, H=Hind III, S=Sac I X=Xho I, C=Cla I, EV=Eco RV restriction endonuclease cleavage sites. WT=wild-type, TV=targeting vector, TL=Targeted locus. Arorwheads with LoxP indicate the sequence Cre mediated recombination.

FIG. 10B: A Southern blot showing Bmx gene analysis in ES cell clones after electroporation and drug selection. The wild-type and gene-targeted DNA hybridization signals are indicated in the figure. WT indicates the migration of the wild type fragment, Targeted indicates the gene targeted DNA fragment.

### DETAILED DESCRIPTION

The growth and differentiation of endothelial cells is regulated by signal transduction through tyrosine protein kinases. We have discovered that the novel cytoplasmic tyrosine kinase gene, Bmx (Bone Marrow tyrosine kinase gene in chromosome X), originally identified in human bone marrow RNA and found to be expressed predominantly in hematopoietic progenitor and myeloid hematopoietic cell lineages, is also highly expressed in human heart endocardium and, importantly, in the endothelium of large arteries. This TK shows unique specificity of expression among tyrosine kinase genes and is involved in signal transduction in endocardial and arterial endothelial cells (Ekman et al. (1997) Circulation 96(6):1729-1732, the disclosure of which is herein incorporated by reference).

Vascular endothelial physiology and pathology, such as arteriosclerosis is largely determined by a receptor-mediated regulation of endothelial cell functions, with associated pathology of the adjacent smooth muscle cell layer. Regulation of Bmx has effects on vascular endothelial cells through Bmx's effect on signal transduction via endothelial cell receptors.

Regulation of vascular endothelial function and inhibition of the development of arteriostenosis such as arteriosclerosis according to the instant invention can be achieved by regulating endogenous Bmx expression in endothelial cells via the Bmx signaling pathway and/or promotor. Alternatively, Bmx expression can be enhanced by stably or transiently incorporating Bmx DNA or RNA into arterial endothelial cells or decreased by transfecting antisense DNA into endothelial cells. The best way, however, to regulate Bmx function is by use of specific tyrosine kinase inhibitors, such as those described for the PDGF and VEGF receptors (Strawn et al. (1996) Cancer Research 56(15):3540-5; Kovalenko et al. (1997) Biochemistry 36(21):6260-9).

In addition to tyrosine kinase inhibitors, small molecular weight inhibitors of signal transduction, such as Wortmannin, which inhibits phosphoinositide 3-kinase can be used to inhibit hemopoietin-cytokine receptor, receptor tyrosine kinase and G-protein coupled receptor signaling (Levitzki (1997) Medical Oncology 14(2):83-9). Such compounds and others can be useful in inhibiting the coupling of receptors to cytoplasmic tyrosine kinases either directly or via docking proteins or intermediate enzymes, which catalyze steps important for the transduced signals. Furthermore, the elucidation and structural analysis of the protein-protein interactions of tyrosine kinases using e.g. the yeast two-hybrid system and crystal structure determination of the isolated domains such as Bmx will make it possible to devise additional pharmacological inhibitors.

Endothelial cells can be modified (e.g., by transfection) to express incorporated genetic material such as naturally occurring or non-naturally occurring/modified BMX genes to produce the encoded product at levels sufficient to produce the normal physiological effects of the Bmx protein if that is desired, or to inhibit (e.g., through antisense) endogenous production of Bmx. The incorporated genetic material may encode a selectable marker, thus providing a means by which cells expressing the incorporated genetic material are identified. Endothelial cells containing incorporated genetic material are referred to as transduced endothelial cells.

Any method or vector suitable for transfection of endothelial cells can be used, e.g., as in Mulligan et al., U.S. Pat. No. 5,674,722, herein incorporated by reference. For example, viral or retroviral vectors have been used to stably transduce endothelial cells with genetic material which includes genetic material encoding a polypeptide or protein of interest not normally expressed at biologically significant levels in endothelial cells. Using, e.g., a retroviral vector, the Bmx mRNA or antisense can be controlled by a retroviral promoter. Alternatively, retroviral vectors having additional promoter elements (in addition to the promoter incorporated in the recombinant retrovirus) which are responsible for the transcription of the Bmx gene, can be used. For example, a construct in which there is an additional promoter modulated by an external factor or cue can be used, making it possible to control the level of polypeptide being produced by the endothelial cells by activating that external factor or cue. Transduction performed in vivo involves applying the recombinant retrovirus encoding Bmx sense or antisense DNA to the desired endothelial cells by, e.g., site directed administration of recombinant retrovirus into a blood vessel via a catheter. Alternatively, endothelial cells that have been transduced in vitro can be grafted onto a blood vessel in vivo through the use of a catheter. The isolation and maintenance of endothelial cells from capillaries and large vessels (e.g., arteries, veins) of many species of vertebrates have been well described in the literature. For example, McGuire and Orkin describe a simple procedure for culturing and passaging endothelial cells from large vessels of small animals. McGuire and Orkin (1987) Biotechniques, 5:546-554.

The practice of the present invention generally employs conventional techniques of molecular biology, microbiology, recombinant DNA, and immunology, which are well within the skill of the art. Such techniques are explained fully in the literature. See for example J. Sambrook et al, “Molecular Cloning; A Laboratory Manual (1989); “DNA Cloning”, Vol. I and II (D. N Glover (ed.) 1985); “Oligonucleotide Synthesis” (M. J. Gait cd, 1984); “Nucleic Acid Hybridization” (B. D. Hames & S. J. Higgins eds. 1984); “Transcription And Translation” (B. D. Hames & S. J. Higgins eds. 1984); “Animal Cell Culture” (R. I. Freshney ed. 1986); “Immobilized Cells And Enzymes” (IRL Press, 1986); B. Perbal, “A Practical Guide To Molecular Cloning” (1984); the series, “Methods In Enzymology” (Academic Press, Inc.); “Gene Transfer Vectors For Mammalian Cells” (J. H. Miller and M. P. Calos eds. 1987, Cold Spring Harbor Laboratory); Meth Enzymol (1987) 154 and 155 (Wu and Grossman, and Wu, eds., respectively); Mayer & Walker, eds. (1987), “Immunochemical Methods In: Cell And Molecular Biology” (Academic Press, London); Scopes, and “Handbook Of Experimental Immunology,” volumes I-IV (Weir and Blackwell, eds, 1986). The disclosures of all of the above references are incorporated herein by reference.

The present invention also encompasses pharmaceutical compositions which include a Bmx tyrosine kinase regulating agent identified using the above-described methods. These compositions include a pharmaceutically effective amount of the agent in a pharmaceutically acceptable carrier or diluent. Acceptable carriers or diluents for therapeutic use are well known in the pharmaceutical art, and are described, for example, in Remington's Pharmaceutical Sciences. 18th ed., Gennaro, ed., Mack Publishing Company, Easton, Pa., 1990, herein incorporated by reference. Preservatives, stabilizers, dyes and even flavoring agents may be provided in the pharmaceutical composition. For example, sodium benzoate, sorbic acid and esters of p-hydroxybenzoic acid may be added as preservatives. In addition, antioxidants and suspending agents may be used.

Compositions containing agents for use in the present invention may be formulated and used as tablets, capsules or elixirs for oral administration, suppositories for rectal administration, sterile solutions, suspensions for injectable administration, and the like. The formulations of this invention can be applied for example by parenteral administration, intravenous, subcutaneous, intramuscular, intracranial, intraorbital, ophthalmic, intraventricular, intracapsular, intraspinal, intracisternal, intraperitoneal, intranasal, aerosol, or oral administration. Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. If desired, absorption enhancing preparations (e.g., liposomes) may be utilized.

The pharmaceutically effective amount of the composition required as a dose will depend on the route of administration, the type of animal being treated, and the physical characteristics of the specific animal under consideration. The dose can be tailored to achieve optimal efficacy but will depend on such factors as weight, diet, concurrent medication and other factors which those skilled in the medical arts will recognize. Typically, human clinical applications of products are commenced at lower dosage levels, with dosage level being increased until the desired effect is achieved. The determination of effective dosage levels, that is the dosage levels necessary to achieve the desired result, will be within the ambit of one skilled in the art based on generally accepted protocols for clinical studies.

In practicing the methods of the invention, the Bmx tyrosine kinase regulating agents can be used alone or in combination with one another, or in combination with other therapeutic or diagnostic agents, employing a variety of dosage forms.

### EXAMPLE 1

### Isolation and Analysis of Mouse Bmx cDNA Clones for in situ Hybridization

Mouse Bmx cDNA was isolated, sequenced and found to encode a protein approximately 91% identical with the human Bmx tyrosine kinase. Northern blotting and in situ hybridization of sections indicated that Bmx mRNA is specifically expressed in the endocardium of the developing heart, endocardium of the left ventricle in adults and in the endothelium of large arteries. Approximately 1×10⁶ bacteriophage lambda clones from a 12 day p.c. mouse embryo cDNA library (Novagen) were screened with a radiolabeled Bam HI fragment (nt 192-1831) of human Bmx cDNA (sequence accession number X83107). One positive clone containing about 1.7 kb including or containing the open reading frame and 3′ untranslated sequence as well as a polyA sequence, was isolated and subcloned as three fragments, which were sequenced from both strands. The remaining 5′ portion of the cDNA was obtained by isolating the first Bmx coding exon from a mouse genomic DNA library in the lambda FIXII vector (Stratagene), using a PCR fragment containing human Bmx nucleotides 23 to 162 as a probe. Primers were designed on the basis of the obtained sequence for PCR amplification of the remaining part of mouse cDNA using mouse heart Quick-Clone cDNA (Clontech) as the template. The PCR reaction conditions were: denaturation at 94° C. for 60 s, annealing at 50° C. for 30 s and extension at 72° C. for 30 s, for 30 cycles in a reaction volume of 50 μl. The PCR fragment obtained was subcloned into the pCRII vector (Clontech) and sequenced. Two independent amplifications and clonings were carried out from the same cDNA using the Dynazyme polymerase (Finnzymes).

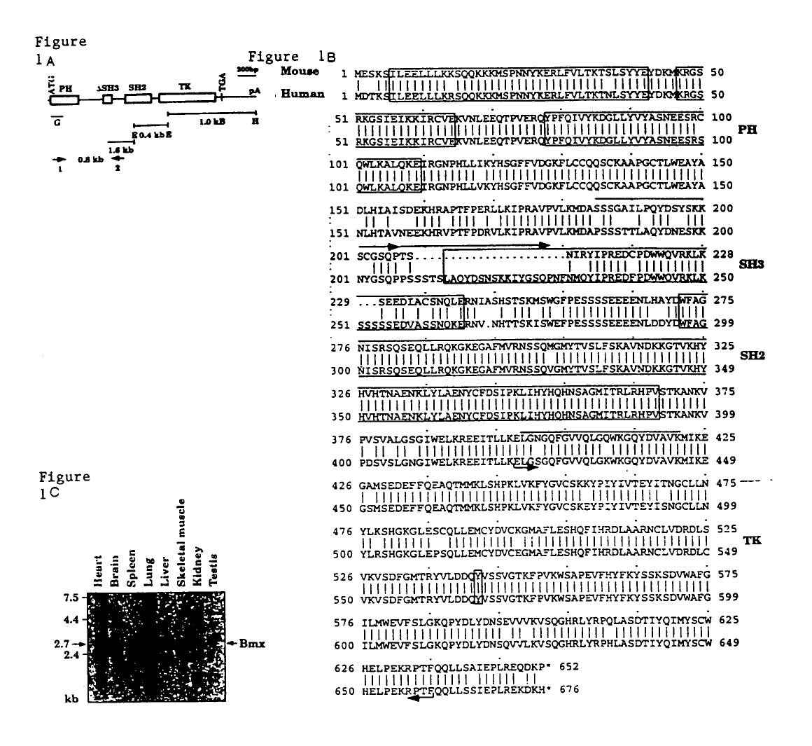

The mouse Bmx cDNA structure and the isolated clones are shown schematically in FIG. 1A. The human BMX cDNA is shown in SEQ. ID NO.: 1. The location of the different protein domains encoded by the cDNA as well as translational start and stop codons and polyadenylation signal are marked in the figure. A comparison of the deduced amino acid sequences of mouse (SEQ. ID. NO.: 2) and human Bmx (SEQ. ID. NO.: 3) genes is shown in FIG. 1B. Comparison of the degree of sequence identity with other members of the Btk/Emt/Tec/Txk/Bmx TK family allowed an unequivocal identification of the clone as the homologue of human Bmx (data not shown).

### EXAMPLE 2

### Analysis and Localization of Bmx mRNA Expression in Tissues

A Northern blot containing 2 μg of polyadenylated RNAs from various mouse tissues (Clontech) was hybridized with the Bmx cDNA fragment probe and washed under stringent conditions, according to the manufacturer's instructions.

The mouse Bmx antisense and sense RNA probes were synthesized from linearized pBluescript II SK+plasmid (Stratagene, La Jolla, Calif.), containing a Hind III-EcoRI fragment from mouse Bmx cDNA (nucleotides 1302-2369; Genbank accession number X83107, by incorporation of [35S]-UTP using T7 and T3 polymerases after linearization with Eco RI and Hind III, respectively. In situ hybridization of paraffin sections was performed as previously described (Kaipainen et al. (1993) J Exp Med 178:2077-2088).

When mouse Bmx cDNA was used to probe a Northern blot containing polyA+RNA from various mouse tissues, clearcut mRNA signals were obtained only from the heart and lung (FIG. 1C).

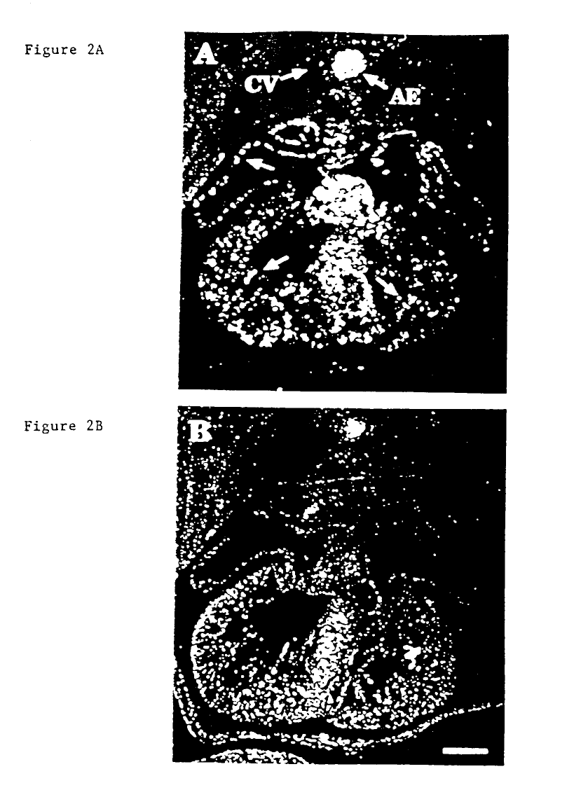

Sections of mouse embryos and adult tissues were processed for in situ hybridization using mouse Bmx cDNA as the probe. The 8.5 day and 9.5 day mouse embryos were negative for Bmx mRNA. In 10.5 and 12.5 day p.c. mouse embryos the Bmx autoradiographic signals decorated the heart endocardium (FIG. 2). Both ventricular and atrial endocardium were positive for Bmx mRNA. In addition, the endothelium of the dorsal aorta showed a strong hybridization signal, whereas the cardinal vein was negative. No other cells were positive for Bmx mRNA in the embryonic sections.

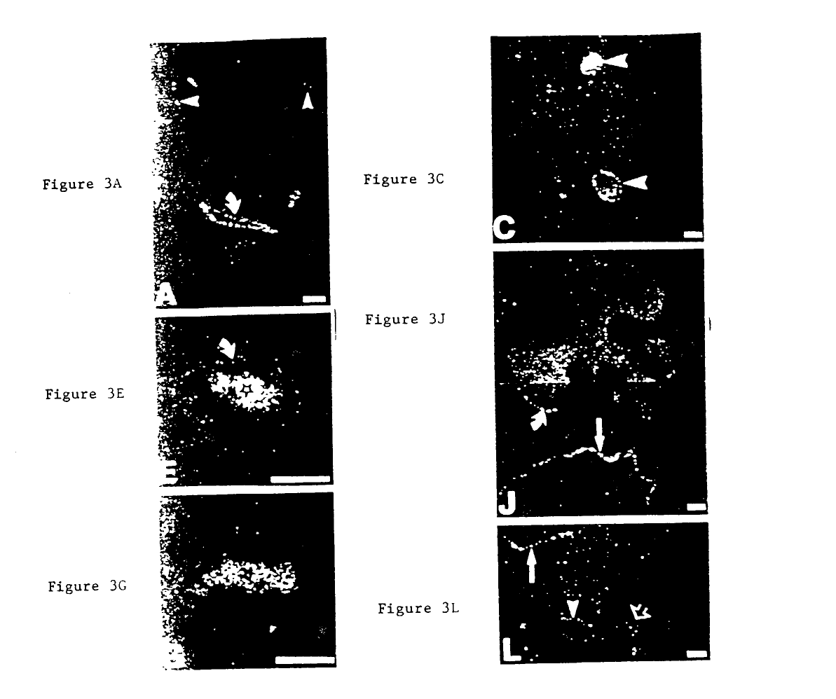

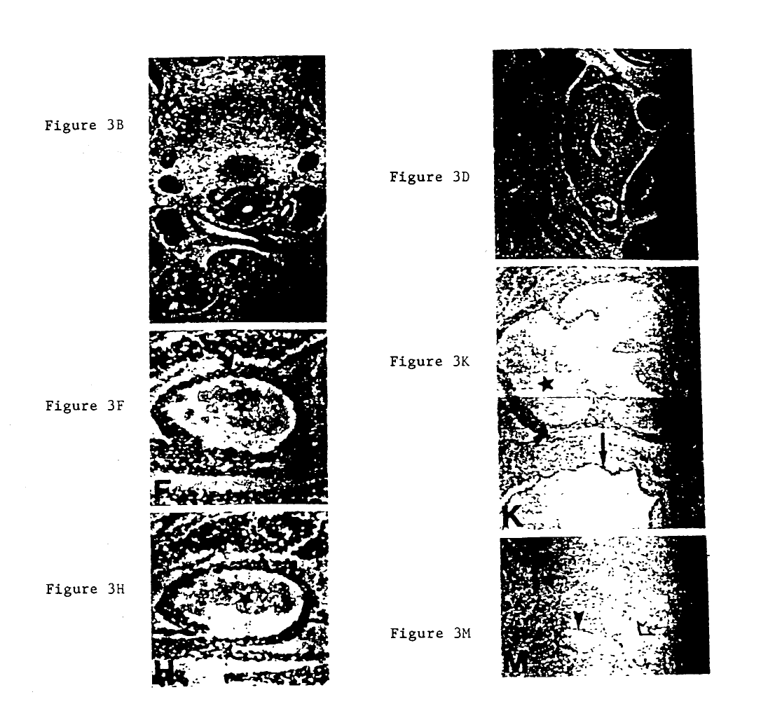

In transverse sections of the thoracic cavity, strong Bmx mRNA signals were also present in the aortal endothelial cells and subclavian arteries as well as in the intervertebral arteries (FIGS. 3A and B). Signal was seen also in the umbilical arteries (C, D). Such signals were considerably weaker, but persisted, in the aortic endothelium of 16.5 day mouse embryos (E, F). The Bmx sense probe did not give a signal in any of these sections (G,H).

Adult mouse lung and kidney were negative for Bmx mRNA in situ hybridization, but autoradiographic signals were obtained from the heart endocardium of the left ventricle and from aorta (J,K). Interestingly, the right ventricular endocardium was negative (data not shown) and also the coronary arteries showed a weak but definitive hybridization signal (L,M).

These data show for the first time expression of the Bmx tyrosine kinase, a member of the Btk/Emt/Tec/Txk/Bmx tyrosine kinase family, outside the hematopoietic system. We have previously shown that the Bmx gene is expressed in bone marrow cells, CD34+hematopoietic cells from umbilical cord blood and in peripheral blood granulocytes (Tamagnone et al. (1994) Oncogene 9:3683-3688, herein incorporated by reference; Kaukonen et al. (1996) B J Haematol 94:455-460). Although the Tec TK has been reported to be expressed in hepatocytes and hepatomas, (Mano et al. (1990) Oncogene 5:1781-1786) previous studies have indicated that the Btk/Emt/Tec/Txk/Bmx TKs function mainly in certain lineages of hematopoietic cells, where they are activated by several upstream signal transducers (Vihinen and Smith, supra).

In addition, recent data indicate that the PH domain of Btk interacts with specific phospholipids, (Tsukada et al. (1994) Proc Natl Acad Sci USA 91:11256-11260) and such binding may be modulated by lipid kinases and phosphatases activated during receptor mediated signal transduction in these cells. In addition, a number of cytokine receptors including c-kit, GCSF, interleukin-3 and erythropoietin receptors were shown to increase tyrosine phosphorylation of Btk/Tec type of kinases (Miyazato et al., (1995) Oncogene 11:619-625; Rawlings and Witte, (1995) Seminars in Immnunology 7:237-246; Miyazoto et al., (1996) Cell Growth and Differentiation 7:1135-1139; herein incorporated by reference). In addition, thrombin receptor, which is coupled to G-protein mediated signal transduction was shown to stimulate phosphorylation of the Tec kinase related to Bmx in platelets (Hamazaki et al. (1998) Oncogene 16:2773-2779; herein incorporated by reference). Recent experiments have indicated that one of the downstream components of the Bmx signal transduction pathway is the Stat transcription factor (Saharinen et al. (1997) Blood 11:4341-4353).

The expression of the Bmx gene apparently begins around day 9.5-10.5 of mouse embryonic development, but it was not restricted to embryonic tissues. BMX transcripts were also identified in adult mouse heart and lung by Northern hybridization. On the basis of the present in situ hybridization results, the mRNA signal in the lung sample is derived from the large arteries present in this material. The signal in the heart sample was considered to be derived from the adult endocardium. Interestingly, the coronary arteries also showed a weak, but definitive BMX mRNA signal.

### EXAMPLE 3

### The Bmx Tyrosine Kinase is Regulated by Vascular Endothelial Growth

### Factor Receptor (VEGFR) and Cytokine Receptors

Materials and Methods:

The polyclonal antibody (Ab) against human Bmx was produced against the Tec Homology (TH) domain by Dr. Toshio Suda (amino acids 151-169, NH2-CNLHTAVNEEKHRVPTFPDR-COOH (SEQ. ID. NO.: 4)). The monoclonal anti-hemagglutinin (HA)-epitope Ab, phosphotyrosine Ab, and anti-CD31 Ab were from Berkeley Antibody Company (Richmond, Calif.), from Transduction Laboratories and from DAKO, respectively. GCSF growth factor was a kind gift from Dr. Riitta Alitalo, while VEGF was obtained from R&D Systems, Minneapolis, Minn. VEGF-B was produced in Drosophila cells by Terhi Karpanen. Mouse IL-3 was obtained from Calbiochem Novabiochem. Phosphatidyl inositol-3 kinase inhibitors Wortmannin and LY294002, were both from Sigma. For inhibition of PI3K activity, 100 nM for 30 min of Wortmannin and 100 μM of LY294002 for 30 min, were used.

DNA constructs were made as follows. Carboxy terminus HA tagged human full-length Bmx cDNA was cloned into the Mlu I-Sal I sites of the pCI-neo expression vector (Saharinen et al. 1997 Blood 11:4341-4353). The kinase dead form of human Bmx, BmxHA K444R, was generated by site specific mutagenesis, using GeneEditor (Promega) kit, 5′-TGTTGCTGTTAGGATGATCAAGG-3′ (SEQ. ID. NO.: 5) as primer and human full-length Bmx cDNA in pCI neo expression vector, as template.

Human GCSFR cDNA cloned into the pEF-BOS expression vector was a kind gift from Dr. Shigekazu Nagata via Dr. Judith Layton. The cDNAs for human VEGFR-1 and VEGFR-3 were cloned into pcDNA3.1Z+ (Invitrogen). The expression plasmid for VEGFR-2 was a kind gift from Dr. Bruce Terman, while the cDNA for EpoR, cloned into pRK5 expression vector, was a gift from Dr Olli Silvennoinen.

Cell culture and transfections. 293T cells were grown in Dulbecco's Modified Eagle's Medium (DMEM), Human umbilical vein endothelial cells (HUVECs) were maintained in Hy-clone 199 medium. 32Dcl3 cells, a gift from Dr. Olli Silvennoinen, were maintaied in RPMI 1640 medium, containing 2 ng/ml mouse IL-3. All media were supplemented with 10% fetal calf serum, glutamine and antibiotics, the Hy-clone 199 medium additionally with endothelial cell growth supplement-ECGS (Upstate Biotechnology). Where indicated, cells were stimulated with growth factors, hGCSF 100 ng/ml, hVEGF and VEGF-B 60 ng/ml, for 5 min or with mIL-3 or hIL-3, 200 ng/ml, for 30 min.

Transfections and generation of stable BmxHA and BmxHA K444R expressing pools (and clones). Transient co-transfections of 293T cells were done using the Calcium Phosphate Transfection kit (GIBCO) according to manufacturer's instructions. The cells were transfected with Bmx-HA expression plasmid and the receptor plasmid or empty vector (PCI-Neo or pcDNAZ3.1) in the ration of 1:4. 30 h after transfection cells were switched to serum-free medium containing 0.2% BSA and the next day lysed in PLCLB or TKB kinase assay lysis buffer. Stable BmxHA and BmxHA K444R expressing 32Dcl3 cells were generated by electroporation (240 mV, 960 μF) followed by selection in 500 μg/ml neomycin.

Immunoprecipitation and Western blotting were performed as follows. The cells were lysed in PLCLB lysis buffer (50 mM HEPES, 150 mM NaCl, 10% Glycerol, 1% Triton X-100, 1.5 mM MgCl2) supplemented with aprotinin, leupeptin, phenylmethylsulfonyl fluoride (PMSF) and sodium vanadate. Equal amounts of protein from cell lysates were used for the immunoprecipitation. Protein concentrations of the lysates were measured using the BioRad Protein Assay system (Bio-Rad Laboratories, Hercules, Calif.). Immunoprecipitation of the lysates was performed by incubation with indicated antibody for 1 to 2 hours followed by binding to protein-G-Sepharose (Pharmacia) or protein-A-Sepharose (Sigma) for 30 min to 1 hour with gentle agitation. The immunoprecipitates were washed, eluted in Laemmli buffering, electrophoresed in 7.5% SDS-PAGE and blotted onto a nitrocellulose filter. Immunodetection was performed using specific primary antibodies and HRP-conjugated anti-mouse or anti-rabbit secondary antibodies (Dako) followed by ECL detection (Amersham).

In vitro substrate immunocomplex kinase assay was performed as follows. The cells were lysed in TKB lysis buffer (1% NP-40, 20 mM Tris-HCl (pH 7.5), 150 mM NaCl, 5 mM EDTA), supplemented with aprotinin, leupeptin, PMSF and sodium vanadate. Immunoprecipitation was performed as described above. After precipitation, the immunoprecipitates were washed two times with lysis buffer, one time with wash buffer (150 mM NaCl, 20 mM HEPES pH 7.4) and two times with kinase assay buffer (10 mM HEPES pH 7.4, 5 mM MnCl₂, sodium vanadate), after which they were resuspended in 5 μl of reaction buffer (1×kinase assay buffer, 5 μM unlabeled ATP, 3 μCi gamma-ATP, 2.5 μg poly (Glu, Tyr) (Sigma)). The kinase reactions were carried out at 30° C. for 10 min and stopped by adding 50 μl of stopping buffer (4 mM unlabeled ATP, 40 mM EDTA, 20 mM HEPES pH 7.4, 100 μg BSA). The sepharoses were spun down and the substrate containing supernatants were spotted on Whatman 3MM filter papers. The filters were fixed and washed one time in 10% TCA 8% sodium pyrophosphate, 3 times in 5% TCA and 2 times in ethanol after which radioactivity was measured using Pharmacia Wallac 1410 Liquid Scintillation Counter. For detection of autokinase activity, the sepharose was eluted in Leammli buffer, and separated in 7.5% SDS-PAGE. The gel was dried and Bmx autokinase activity was detected by exposure on film.

Results:

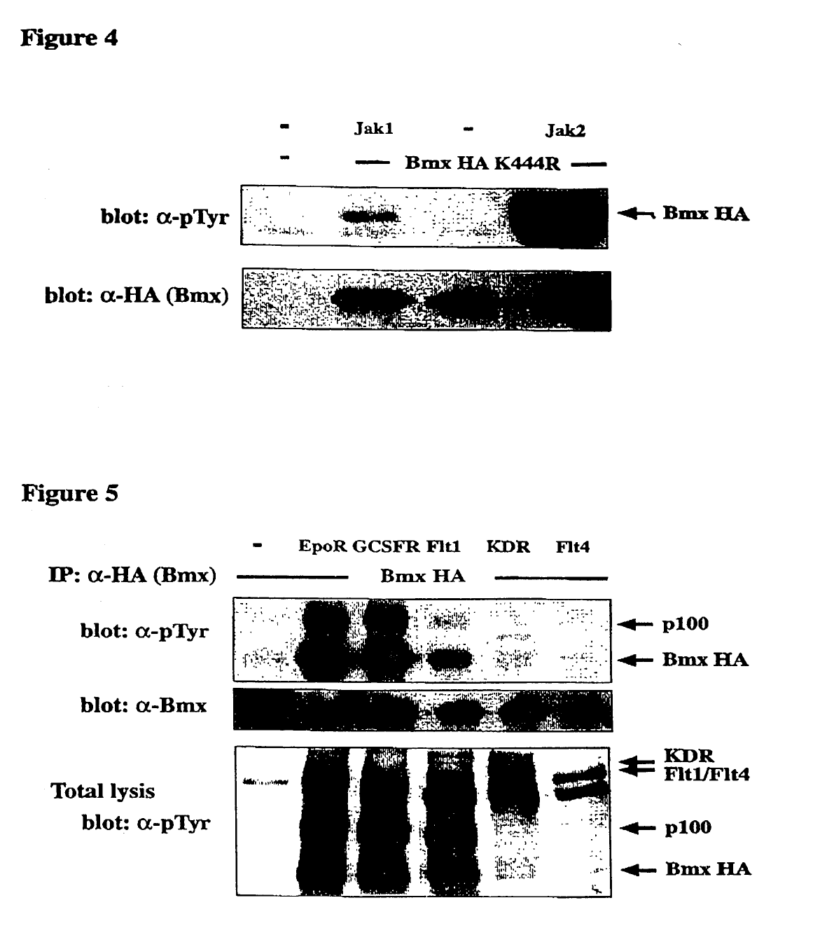

Phosphorylation of kinase dead Bmx by Jak kinases. Kinase dead Bmx was coexpressed with Jak1 or Jak2 in 293T cells. FIG. 4 shows that Jak2, and in a lesser extent, Jak1 phosphorylate kinase dead Bmx.

EpoR, GCSFR and Flt1 induce Bmx phosphorylation in 293T cells (FIG. 5). When Bmx was coexpressed in 293T cells together with EpoR or GCSFR, a clear phosphorylation of Bmx could be detected. Also Flt1 phosphorylated Bmx, although in a smaller amount, while no increase in phosphorylation level could be detected after coexpression with KDR or Flt4. A phosphorylated protein of approximately 100 kD was co-precipitated with the phosphorylated Bmx. All cells were starved in 0.2% BSA over night before lysis. Note that exposure time for Flt1, KDR and Flt4 total lysates are longer than to the corresponding other receptors, in order to detect these receptors.

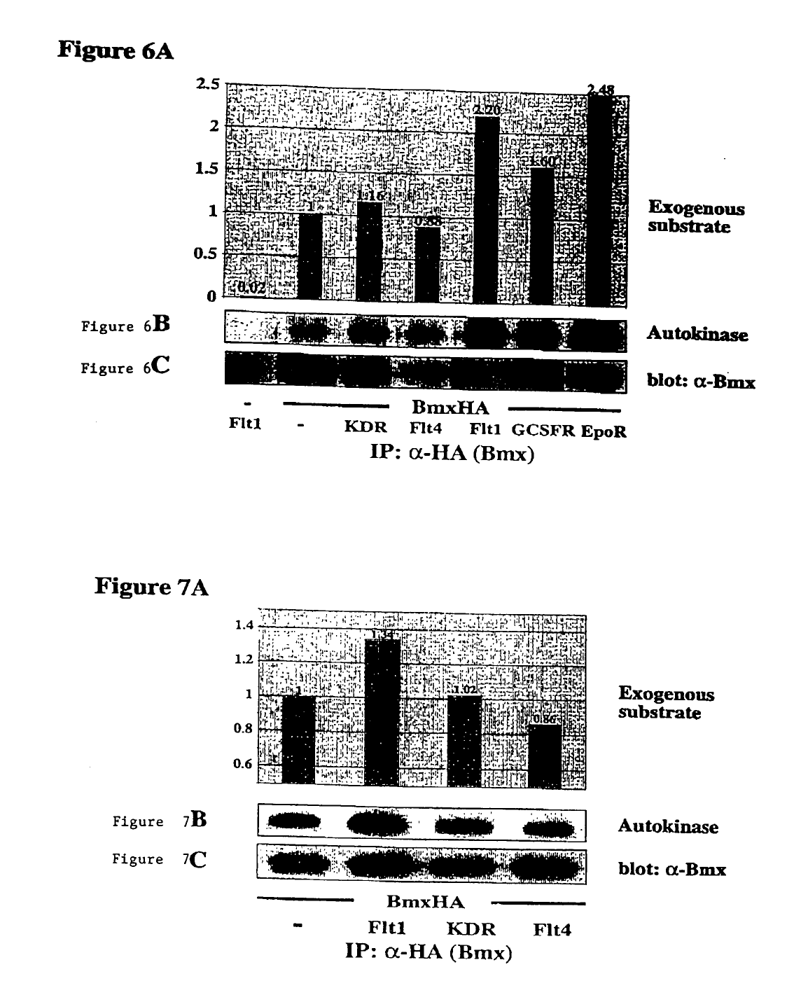

Coexpression of Bmx and VEGFR-1, EpoR or GCSFR in 293T cells increases kinase activity of Bmx. As can be seen from FIG. 6, when Bmx was coexpressed in 293T cells together with indicated receptors, a clear activation of both substrate (poly (glu-tyr)) kinase activity (FIG. 6A) and autokinase activity (FIG. 6B) could be detected after expression with EpoR, GCSFR and Flt1, while KDR and Flt4 did not activate Bmx. The Bmx levels were same in all reactions as determined by the anti-Bmx blot (FIG. 6C). FIG. 7 shows a comparison of Bmx activity after co-transfection of Bmx and VEGF receptors.

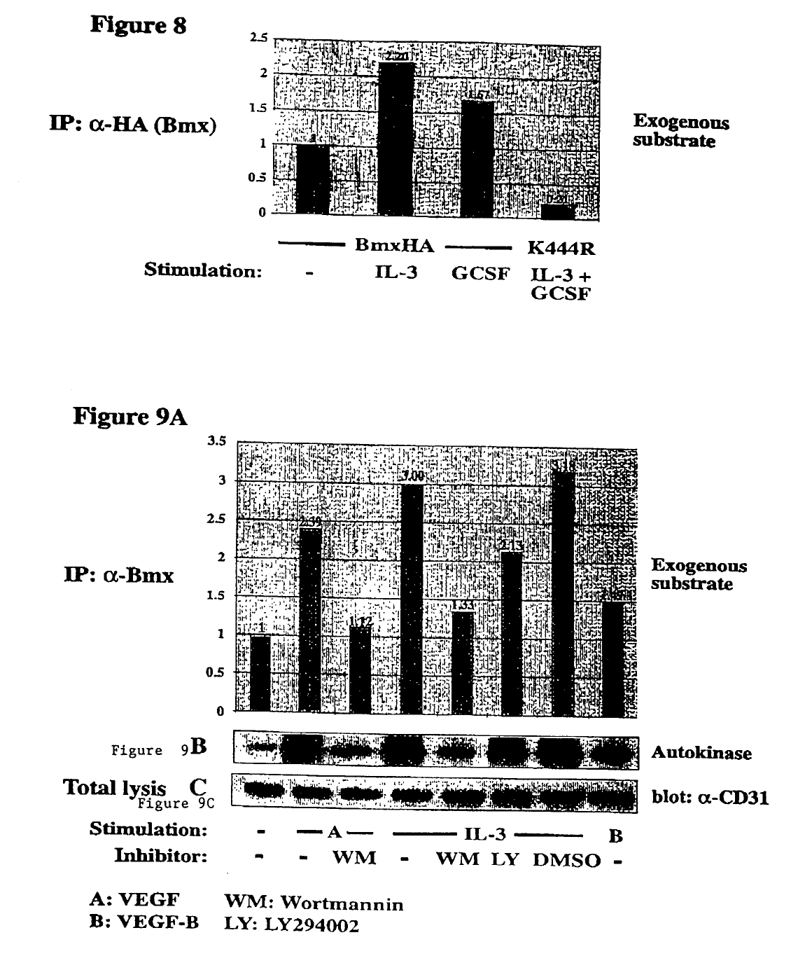

IL-3 and GCSF increase Bmx kinase activity in 32Dcl3 cells using endogenous IL-3 and GCSF receptors. In FIG. 8, BmxHA or kinase dead (K444R) Bmx expressing 32Dcl3 pools, were stimulated with IL-3, GCSF or were left unstimulated. IL-3 increased the kinase activity of Bmx about 2.2 fold, while the increase after GCSF stimulation was around 1.7. Kinase dead mutation of Bmx only showed background activity. The same amount of total protein (120 μg) was used for all immunoprecipitations. Cells were starved in IL-3 and serum free media for 14 hours before lysis.

Activation of endogenous Bmx. Human Umbilical Vein Endothelial Cells (HUVECs) were stimulated with VEGF, VEGF-B or IL-3, and kinase activity of Bmx was measured. FIGS. 9A and B show that stimulation with Flt1 activating factors, VEGF and VEGF-B, increased endogenous Bmx activity in the same way as in transfected 293T cells. As in 32Dcl3 cells, also IL-3 increased Bmx kinase activity in HUVECs. PI3K inhibitors Wortmannin, and to a less extence LY294002 (2-(4-morpholinyl)-8-phenyl-4H-1-benzopyran-4-one), were able to block the activation. FIG. 9C, control blotting with endothelial specific anti-CD31 to confirm equivalent amounts of total protein in each reaction.

Together, these results show that the receptors for erythropoietin, vascular endothelial and granulocyte growth factors activate Bmx tyrosine kinase in conditions of overexpression in 293T cells. The receptors for vascular endothelial growth factors 2 and 3 were not able to activate Bmx. The overexpressing conditions in 293T cells presumably activate the receptors similar to ligand binding. In addition, endogenous Bmx in HUVECs was activated after stimulation with VEGF and VEGF-B, both ligands of Flt1 receptor, showing that the activation seen in 293T cells is not due to the overexpression. In HUVECs an inhibition of the activation of Bmx could be detected after use of PI3K inhibitor Wortmannin. Similar results were obtained with LY294009. These results suggest that Bmx is involved in many different types of signal transduction, at least GCSF, VEGF and IL-3 signaling. The activation of Bmx from EpoR is most probably due to the use of Jak2 as an primarily signal transducer of the receptor. Our results so far suggest that Jak tyrosine kinases are important for the activation of Bmx, which could explain the activation detected also from EpoR, even though EpoR and Bmx are not endogenously expressed in the same cells. The increase in Bmx kinase activity after coexpression with GCSFR or after stimulation with GCSF, is highly interesting, because GCSF is the most widely used growth factor for patients whose bone marrow function is compromised. Also the specific activation of Bmx by VEGFR-1 (Flt1) but not from other VEGF receptors in an interesting finding.

### EXAMPLE 4

### Inactivation of the BMX Gene and Generation of BMX Deficient Mice

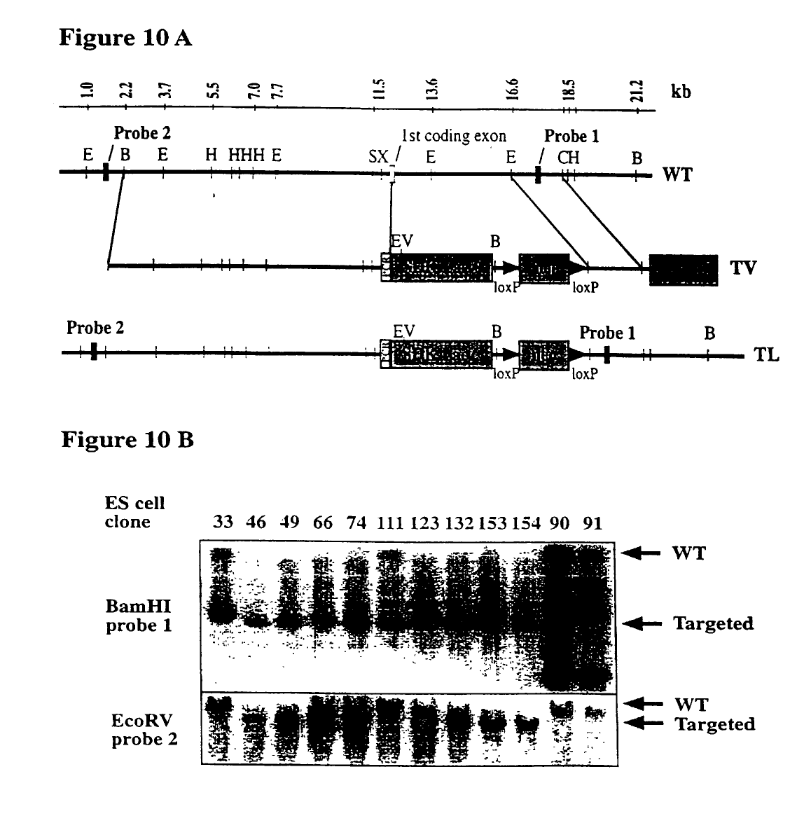

The mouse Bmx gene was cloned from a mouse 129SV genomic DNA library using the human Bmx cDNA fragment consisting of nucleotides 23-162 as the hybridization probe for screening the bacteriophage lambda library. The region surrounding the first coding exon was characterized by restriction digest mapping. Suitable fragments flanking the first coding exon (containing the ATG initiation codon) were isolated and used to construct the targeting vector containing the LacZ and neomycin resistance cassettes employing the strategy described by Puri et al. (Puri et al. 1995 EMBO J. 14: 5884-5891; herein incorporated by reference). For negative selection, the herpes simplex virus tymidine kinase (HSV-tk) casette is included in the 3′ end of the construct. The targeting construct, shown schematically in FIG. 10, replaces the first coding exon containing the ATG initiation codon and should thereby abolish expression of the gene upon homologous recombination in embryonic stem (ES) cells.

Transfection of mouse ES cells with the targeting constructs and screening of single clones by Southern blotting was done using standard procedures. Upon Southern blotting and hybridization using the fragments indicated in FIG. 10A, the successfully targeted clones and Bmx genes could be identified. Probe 1 consists of a 3′ internal XmnI fragment, while probe 2 was a 5′ external XbaI fragment. Analysis of 164 clones indicated that the homolougus transfection efficiency was found to be about 5.5%. Seven positive clones plus two non homologous recombined clones were grown up for aggregation with wild type ES cells in order to generate chimeric mice using methods standard in the art. These mice are screened for germline transmission and mated with wild type mice. Due to the X chromosomal location of Bmx, both heterozygous female mice, as well as nullizygous male mice can be generated.

Collectively, these data show that Bmx is involved in the physiology of endothelial cells located at sites of great fluid shear/pressure stress. Bmx is involved in relaying endothelial cell related signals in endocardial and arterial endothelial cells. Alteration of such signals can be used to lead to a long-term readjustment of gene expression in the affected cells with secondary effects in the surrounding smooth muscle cells and extracellular matrix.

The preceding description has been presented with reference to presently preferred embodiments of the invention. Workers skilled in the art and technology to which this invention pertains will appreciate that alterations and changes in the described embodiments may be practiced without meaningfully departing from the principal, spirit and scope of this invention. Accordingly, the foregoing description should not be read as pertaining only to the precise embodiments described and illustrated in the accompanying drawings, but rather should be read consistent with and as support to the following claims which are to have their fullest and fair scope.

1. A method of treating or preventing arteriostenosis in a patient comprising regulating Bmx tyrosine, kinase activity in endocardial and arterial endothelial cells in a manner sufficient to regulate growth signals in endothelium or adjacent smooth muscle, to regulate inflammatory response, or to improve the non-thrombogenic properties of vascular endothelium.

2. The method of claim 1 wherein the arteriostenosis is selected from the group consisting of arteriosclerosis and vasospasm.

3. The method of claim 1 wherein the Bmx tyrosine kinase activity regulation comprises administering to the patient an effective amount of an agent capable of inhibiting Bmx tyrosine kinase activity.

4. The method of claim 3 wherein the agent comprises a tyrosine kinase inhibitor.

5. The method of claim 3 wherein the agent is administered to the vascular endothelium of the patient.

6. The method of claim 3 wherein the agent comprises antisense BMX cDNA.

7. The method of claim 1 wherein the regulation of Bmx tyrosine kinase activity is in endocardial and arterial endothelial cells located at a site of turbulent vascular flow.

8. The method of claim 1 wherein the Bmx tyrosine kinase activity is regulated by inducing a ligand to bind to a cell surface receptor that is molecularly linked to Bmx.

9. The method of claim 8 wherein the ligand is IL-3 or VEGF.

10. The method of claim 8 wherein the ligand binding induces an increase of Bmx tyrosine kinase activity.

11. The method of claim 10 wherein the increased Bmx tyrosine kinase activity results in tyrosine phosphorylation.

12. The method of claim 4 wherein the tyrosine kinase inhibitor is a protein kinase C.

13. The method of claim 12 wherein the protein kinase C is protein kinase C delta.

14. The method of claim 4 wherein the tyrosine kinase inhibitor is an activator of protein kinase C.

15. The method of claim 14 wherein the activator of protein kinase C is a phorbol ester.

16. The method of claim 1 wherein the Bmx tyrosine kinase activity is regulated via expression of a genetically modified Bmx which is molecularly or functionally distinguishable from naturally occurring Bmx.

17. The method of claim 16 wherein expression of the genetically modified Bmx is operably linked to a promoter that is not a natural promoter of BMX.

18. The method of claim 6 wherein the antisense BMX cDNA comprises a promoter operably linked to the antisense cDNA.

19. A method of inhibiting a thrombotic, mitotic, or inflammatory effect in vascular endothelium comprising regulating Bmx tyrosine kinase activity in endocardial and arterial endothelial cells.

20. The method of claim 19 wherein the effect to be inhibited is the result of the influence of at least one of the compounds selected from the group thrombin, IL-3 and VEGF.

21. A method of identifying agents which affect a Bmx tyrosine kinase signaling pathway comprising applying a test agent to tissue from a transgenic animal or administering the test agent to the transgenic animal, the cells of the animal having a genetic defect in the Bmx encoding region, the defect causing an abnormal Bmx signaling pathway, wherein an agent affecting the Bmx signaling pathway is one which functionally restores at least one step in the abnormal pathway.

22. A method for diagnosing a human disease associated with Bmx dysfunction due to mutation in the BMX gene on chromosome X of a patient comprising assaying the BMX gene on the X chromosome of a patient and analyzing the result of the assay to detect the mutation.

23. A method of accelerating re-endothelialization of damaged arterial or endocardial walls comprising regulation of Bmx tyrosine kinase activity in endocardial and arterial endothelial cells in a manner sufficient to enhance growth signals in the endothelium.
